(19) United States
(12) Patent Application Publication
(10) Pub. No.: US 2026/0324952 A1
(43) Pub. Date: Oct. 22, 2026

(54) INSTRUMENT ENGAGEMENT TOOL FOR AN ENDOSCOPE

(71) Applicant: Boston Scientific Scimed, Inc., Maple Grove, MN (US)

(72) Inventors: Alexander Cohen, Austin, TX (US); Luis Amador, JR., Pflugerville, TX (US)

(73) Assignee: Boston Scientific Scimed, Inc., Maple Grove, MN (US)

(21) Appl. No.: 19/651,187

(22) Filed: Apr. 17, 2026

Related U.S. Application Data
(60) Provisional application No. 63/790,850, filed on Apr. 18, 2025.

(51) Int. Cl. A61B 1/00; A61B 1/018

(52) U.S. Cl. CPC A61B 1/00128; A61B 1/018

(57) ABSTRACT

Devices, systems, and methods for hub for use with an endoscope. An example hub configured for use with an endoscope may include a housing and a locking member. The housing may define a channel configured to be in communication with a working channel of the endoscope and configured to receive one or more instruments. The locking member may be in communication with the channel and configured to adjust between a first configuration and a second configuration. The locking member may be configured to engage an instrument of the one or more instruments and prevent longitudinal movement of the instrument when the locking member is in the second configuration. The locking member may be configured to allow the instrument to adjust longitudinally when the locking member is in the first configuration.

### CROSS-REFERENCE TO RELATED APPLICATIONS

This application claims the benefit of U.S. Provisional Patent Application Ser. No. 63/790,850 filed on Apr. 18, 2025, the disclosure of which is incorporated herein by reference.

### FIELD

The present disclosure pertains to endoscopes, endoscope assemblies, guide tubes, introducers, and instrument caps for endoscopes, guide tubes, and introducers. More particularly, the present disclosure pertains to tools for engaging instruments used with an endoscope.

### BACKGROUND

A wide variety of endoscope assemblies and accessories for use with endoscopes have been developed. Of the known endoscope assemblies and accessories for use with endoscopes, each has certain advantages and disadvantages. There is an ongoing need to provide alternative endoscope assemblies and accessories for use with endoscopes as well as methods for making and using the same.

### SUMMARY

This disclosure provides design, material, manufacturing method, and use alternatives for medical devices and medical systems. In a first example, a hub configured for use with an endoscope may include a housing defining a channel configured to be in communication with a working channel of the endoscope and receive one or more instruments and a locking member in communication with the channel and configured to adjust between a first configuration and a second configuration. The locking member may be configured to engage an instrument of the one or more instruments and prevent longitudinal movement of the instrument when the locking member is in the second configuration and the locking member may be configured to allow the instrument to adjust longitudinally when the locking member is in the first configuration.

Alternatively or additionally to any of the examples above, the hub may further include a biasing member configured to bias the locking member in the first configuration.

Alternatively or additionally to any of the examples above, the hub may further include a biasing member configured to bias the locking member in the second configuration.

Alternatively or additionally to any of the examples above, the locking member may comprise an opening configured to receive the instrument when the locking member is in the first configuration.

Alternatively or additionally to any of the examples above, the locking member may be configured to receive a second instrument of the one or more instruments in the opening when the locking member is in the first configuration and allow the second instrument to longitudinally adjust when the locking member is in the second configuration.

Alternatively or additionally to any of the examples above, the housing may be configured to releasably couple with a port of the endoscope.

Alternatively or additionally to any of the examples above, the housing may comprise an opening configured to receive a port of the endoscope and axially align the channel with an opening of the port.

Alternatively or additionally to any of the examples above, the hub may further include a biasing member configured to bias the locking member in one of the first configuration and the second configuration, wherein the locking member comprises a lever and is coupled with the biasing member.

Alternatively or additionally to any of the examples above, a first end portion of the locking member may be coupled with the biasing member and a second end portion of the locking member may form the lever configured to receive a force from a user to adjust the locking member from the first configuration to the second configuration.

Alternatively or additionally to any of the examples above, the locking member may have an arc portion extending through the channel.

Alternatively or additionally to any of the examples above, the locking member may extend through the channel and may include an opening in communication with the channel.

Alternatively or additionally to any of the examples above, the opening may have a first central axis aligned with a second central axis of the channel when the locking member is in the first configuration and the first central axis is offset from the second central axis when the locking member is in the second configuration.

Alternatively or additionally to any of the examples above, the hub may further include a seal located in the channel, wherein the seal may be configured to receive the one or more instruments.

In another example, an endoscope may include a handle comprising an instrument port defining an instrument channel configured to receive one or more instruments therein, an elongate shaft extending in a distal direction from the handle and defining a lumen in fluid communication with the instrument channel, and a locking member in communication with the instrument channel and configured to adjust between a first configuration and a second configuration, wherein the locking member is configured to engage an instrument of the one or more instruments and prevent longitudinal movement of the instrument when the locking member is in the second configuration and the locking member is configured to allow the instrument to adjust longitudinally when the locking member is in the first configuration.

Alternatively or additionally to any of the examples above, the endoscope may include a biasing member, wherein the locking member may include a lever coupled with the biasing member and configured to engage the instrument of the one or more instruments when the locking member is in the second configuration.

Alternatively or additionally to any of the examples above, the instrument port may include a hub housing configured to releasably couple with a handle housing coupled with the elongate shaft.

Alternatively or additionally to any of the examples above, the hub housing may be configured to clip onto the handle housing.

In another example, a method may include inserting a first instrument through an opening in a hub of an endoscope when a locking member of the hub is in a first configuration, inserting a second instrument through the opening in the hub, and adjusting the locking member to a second configuration to engage the first instrument with the locking member and prevent longitudinal movement of the first instrument while the second instrument is longitudinally adjustable.

Alternatively or additionally to any of the examples above, the method may include inserting the first instrument and the second instrument through a lock opening in the locking member, wherein the first instrument is engaged by an edge of the opening in the hub and an edge of the lock opening when the locking member is in the second configuration.

Alternatively or additionally to any of the examples above, adjusting the locking member to the second configuration comprises applying a force on the locking member and the method may further include releasing the locking member to cause the locking member to automatically return to the first configuration.

These and other features and advantages of the present disclosure will be readily apparent from the following detailed description, the scope of the claimed invention being set out in the appended claims.

### BRIEF DESCRIPTION OF THE DRAWINGS

The accompanying drawings, which are incorporated in and constitute a part of this specification, illustrate various configurations and together with the description serve to explain the principles of the present disclosure.

FIG. 1 depicts a schematic perspective view of an illustrative endoscope assembly;

FIG. 2 depicts a schematic exploded view of a portion of the illustrative endoscope assembly shown in FIG. 1;

FIG. 3 depicts a schematic perspective view of a portion of an illustrative endoscope assembly;

FIG. 4 depicts a schematic perspective view of an illustrative tool for use with an endoscope;

FIG. 5 depicts a schematic side view of the illustrative tool depicted in FIG. 4;

FIG. 6 depicts a schematic bottom view of the illustrative tool depicted in FIG. 4;

FIG. 7 depicts a schematic top view of the illustrative tool depicted in FIG. 4, with the tool in a first configuration;

FIG. 8 depicts a schematic top view of the illustrative tool depicted in FIG. 4, with the tool in a second configuration;

FIG. 9 depicts a schematic cross-section view of the illustrative tool depicted in FIG. 4, taken along a line 9-9 in FIG. 7;

FIG. 10 depicts a schematic cross-section view of the illustrative tool depicted in FIG. 4, taken along a line 10-10 in FIG. 8; and

FIGS. 11A-11E depict schematic cross-section views of steps in an illustrative method of using an illustrative tool.

While the disclosure is amenable to various modifications and alternative forms, specifics thereof have been shown by way of example in the drawings and will be described in detail. It should be understood, however, that the intention is not to limit the invention to the particular configurations described. On the contrary, the intention is to cover all modifications, equivalents, and alternatives falling within the spirit and scope of the disclosure.

### DETAILED DESCRIPTION

This disclosure is now described with reference to an illustrative medical system that may be used in endoscopic medical procedures. However, it should be noted that reference to this particular procedure is provided only for convenience and not intended to limit the disclosure. The concepts underlying the disclosed devices and related methods of use may be utilized in any suitable procedure, medical or otherwise. This disclosure may be understood with reference to the following description and the appended drawings, wherein like elements are referred to with the same reference numerals.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, the following discussion may apply equally to any and/or all of the components for which there are more than one, unless explicitly stated to the contrary. Additionally, not all instances of some elements or features may be shown in each figure for clarity.

It is noted that references in the specification to “a configuration”, “some configurations”, “other configurations”, etc., indicate that the configuration(s) described may include a particular feature, structure, or characteristic, but every configuration may not necessarily include the particular feature, structure, or characteristic. Moreover, such phrases are not necessarily referring to the same configuration. Further, when a particular feature, structure, or characteristic is described in connection with a configuration, the particular feature, structure, or characteristic may be effected in connection with other configurations, whether or not explicitly described, unless clearly stated to the contrary. That is, the various individual elements described below, even if not explicitly shown in a particular combination, are nevertheless contemplated as being combinable or arrangeable with each other to form other additional configurations or to complement and/or enrich the described configuration(s).

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is illustrative only. In some configurations, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element.

The detailed description is intended to illustrate but not limit the disclosure. The various elements described may be arranged in various combinations and configurations without departing from the scope of the disclosure. The detailed description illustrates example embodiments of the disclosure.

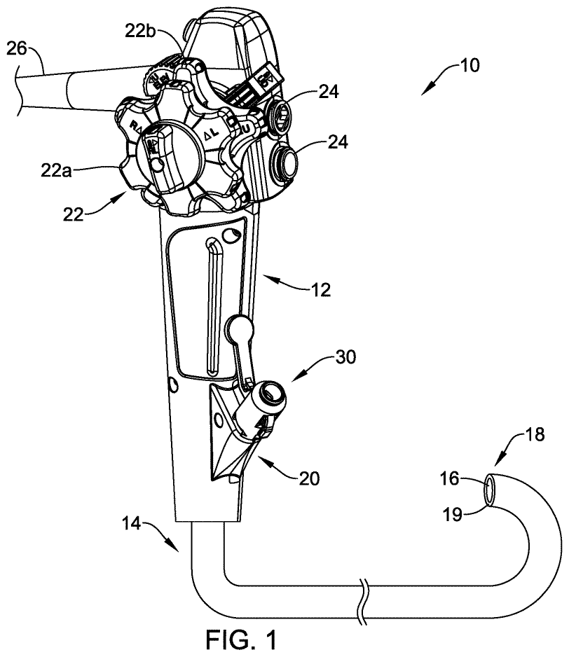

An example endoscope and/or endoscope assembly 10 is illustrated in FIG. 1. The endoscope 10 may be any of a number of types of endoscopes or related medical devices usually identified by the particular anatomy desired to be reached. For example, the endoscope 10 may be a bronchoscope, colonoscope, duodenoscope, esophagoscope, guidetubes, introducers (without or without vision or visualization capabilities), or any other type of endoscope or related medical device. The endoscope 10 may include a handle or handpiece 12 and an elongate shaft 14 extending distally from the handpiece 12 to a distal tip 18. The shaft 14 may include a lumen defining a working channel 16 extending through the shaft 14 from a distal end 19 near the distal tip 18 of shaft 14 to an access port 20 that may be positioned in the handpiece 12 or another portion of the endoscope 10. In some examples, the access port 20 may include or receive a cap or hub 30 configured to receive one or more instruments usable with the endoscope 10. Although the endoscope 10 is depicted with a single working channel in FIG. 1, it can be appreciated that in other suitable configurations, the endoscope 10 may include multiple working channels, as desired.

In some cases, the handpiece 12 may include one or a plurality of controls 22, such as rotating knobs, which may be used to control movement of the distal tip 18 of the shaft 14 during operation. For example, a first rotating knob 22a may control up and down movement or deflection of the distal tip 18 of the shaft 14, while a second rotating knob 22b may control side-to-side movement or deflection of the distal tip 18 of the shaft 14. The handpiece 12 may also include one or a plurality of buttons 24, which may be used to activate suction or deliver fluid such as air, saline and/or water, etc. through a lumen of the endoscope 10 or perform other functions as desired. Additionally, in some cases, the handpiece 12 may include an optical cable 26 connected to an external light source (not shown).

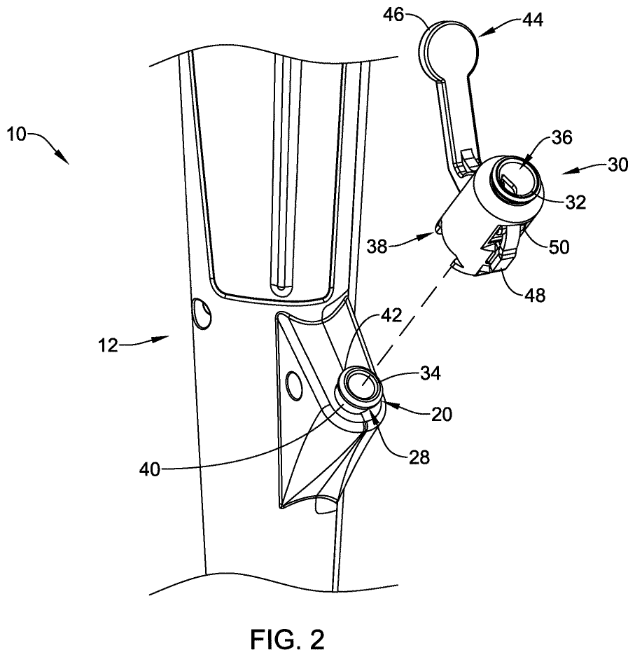

Turning now to FIG. 2, here the access port 20 of the handpiece 12, which provides access to the working channel 16 of the endoscope 10, is illustrated. The access port 20, which may extend from the side of the endoscope 10 or at another suitable location, may include a coupling portion 28 for coupling (e.g., clipping or otherwise connecting) the cap or hub 30 to the access port 20 and an opening 34 in fluid communication with the working channel. The hub 30, which may be removably attached or permanently attached to (e.g., as part of) the access port 20, may provide access for inserting and/or advancing an endoscopic device or instrument through the working channel 16 of the endoscope 10. The hub 30 shown in FIG. 1 may be merely illustrative, as the hub 30 may have one or more of a variety of different exterior profiles.

When the hub 30 is configured to couple with the access port 20, the hub 30 may include a connector 38 configured to couple with the coupling portion 28 of the access port 20, as depicted for example in FIG. 2. The connector 38 may have any suitable configuration designed to couple with the coupling portion 28 of the access port 20. In some examples, when the coupling portion 28 includes a neck 40 and a flange 42 (e.g., an annular flange, as depicted in FIG. 2), the connector 38 may include an opening configured to receive the neck 40 and/or the flange 42 and extend under, around, and/or over the flange 42. In some examples, the connector 38 may be configured to snap or clip onto the coupling portion 28 of the access port 20. Other suitable configurations of the connector 38 are contemplated.

In some cases, caps or hubs like the hub 30, which may be termed “biopsy caps” or “biopsy hubs”, may be designed with several functions in mind. For example, the hub 30 may form a fluid/air barrier to the working channel 16 that may help control insufflation and bile fluid egress therefrom that may later have the potential to spill onto the clinician's hands and/or the floor thereby interfering with the intervention and/or become a biohazard.

As discussed, tools or instruments may be received within the working channel 16 of the endoscope 10 via the access port 20 and/or the hub 30. When one or more instruments are received within the working channel 16 of the endoscope 10, a medical practitioner may hold the handpiece 12 of the endoscope 10 between their arm and their chest, while using their hands to adjust the received instrument(s) and or controls 22 (e.g., the buttons 24, the knobs 22a, 22b, etc.) When it is desired to maintain a longitudinal and/or rotational orientation of the received instrument relative to the working channel 16, a second user (e.g., a third hand) may be needed to maintain the orientation of the instrument while the controls 22 are adjusted and/or one or more other received instruments are adjusted relative to the working channel 16 by a first user (e.g., the medical practitioner).

It may be desirable to lock or secure received instruments or tools in a desired orientation (e.g., rotationally and/or longitudinally lock or secure received instruments) relative to the working channel 16 and/or other suitable portions of the endoscope 10 without the use of a second user and/or while the medical practitioner holds the handpiece 12 with their hands to maintain better control over a position of the endoscope relative to when the handpiece 12 is held between the arm and chest of the medical practitioner. Additionally or alternatively, it may be desirable to adjust (e.g., longitudinally and/or rotationally adjust) one or more instruments received in the access port 20 relative to the working channel 16 with and/or independently of one or more other instruments received in access port 20 and locked or configured to be locked relative the working channel 16 without the use of a second user and/or without holding the handpiece 12 between the chest and arm of the medical practitioner. Illustrative configurations of the hub 30 discussed herein may be configured to receive and selectively lock or secure one or more instruments relative to the working channel 16, while the medical practitioner holds the handpiece 12 with their hands.

To facilitate receiving instruments and/or other tools usable with the endoscope 10, the hub 30 may have an opening 32 in fluid communication with an instrument channel 36 extending through the hub 30. When the hub 30 is part of or is coupled with the access port 20, the opening 32 may be in fluid communication with the working channel 16 and the instrument channel 36. The instrument channel 36 of the hub 30 may include a channel opening 37 and may be configured to receive an instrument and adjust to engage a received instrument within the hub 30. Thus, the hub 30 may be an adapter in that instrument channel 36 of the hub 30 may adjust to engage a received instrument and longitudinally and/or rotationally lock or secure the received instrument relative to the access port 20 and/or the working channel 16.

To facilitate adjusting the hub 30 between a first configuration (e.g., a receiving or unlocked configuration or an engaging or locked configuration) and a second configuration (e.g., the other of the receiving or unlocked configuration and the engaging or locked configuration), the hub 30 may include an actuator 44. The actuator 44 may have any suitable configuration to adjust the hub 30 between the first configuration and the second configuration. In some examples, the actuator 44 may include a lever 46, a biasing member 48, a locking member 50, a button, a spring, an arm, and/or other suitable components as discussed herein or otherwise.

A number of additional caps or hubs are contemplated that incorporate at least some of the features of the hub 30 discussed herein as well as have other desirable characteristics. In some examples, the hub 30 may be or may include a passive seal. For the purposes of this disclosure, a passive seal is a seal that seals the endoscope 10 at or proximate the port 20 so as to prevent the leakage of bodily fluids and/or air. In addition, by virtue of being “passive”, the caps disclosed herein are configured to seal off the endoscope 10 at or proximate the access port 20 without the need of any so-called “active” processes or steps by the clinician. Alternatively or additionally, the hub 30 may be or include an active seal. For example, the seal may be configured to seal of the endoscope 10 at or proximate the access port 20 when an instrument is inserted through the seal.

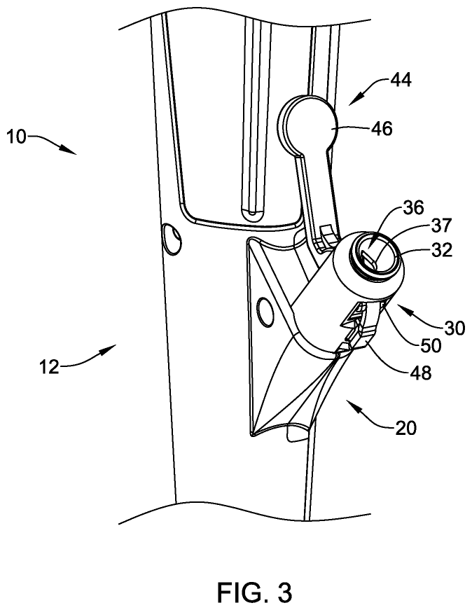

FIG. 3 schematically depicts a perspective view of a portion of the handpiece 12 with the access port 20 and the hub 30. In some examples, the hub 30 may be formed integrally with and as part of the access port 20 and/or the handpiece 12 (as part of a housing of the handpiece 12), as depicted for example in FIG. 3. Other suitable configurations of the hub 30 formed integrally with and as part of the access port 20 and/or the handpiece 12.

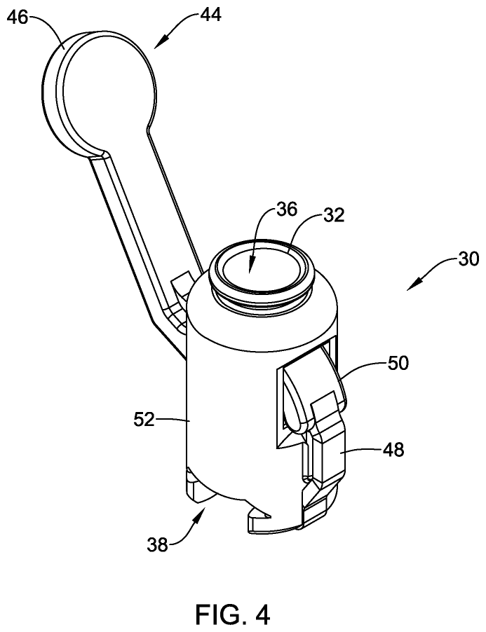

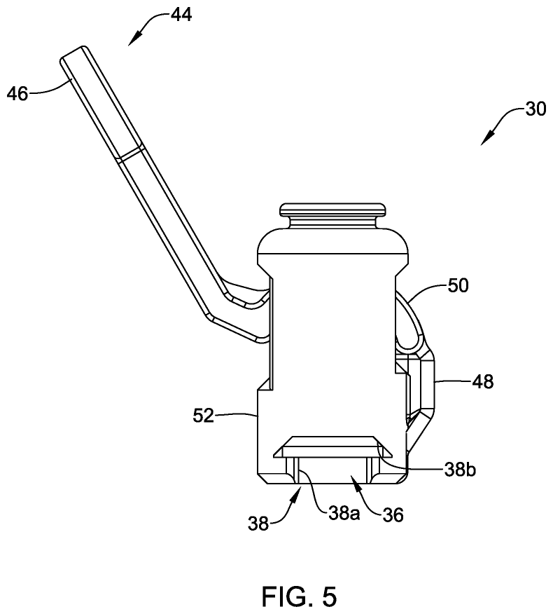

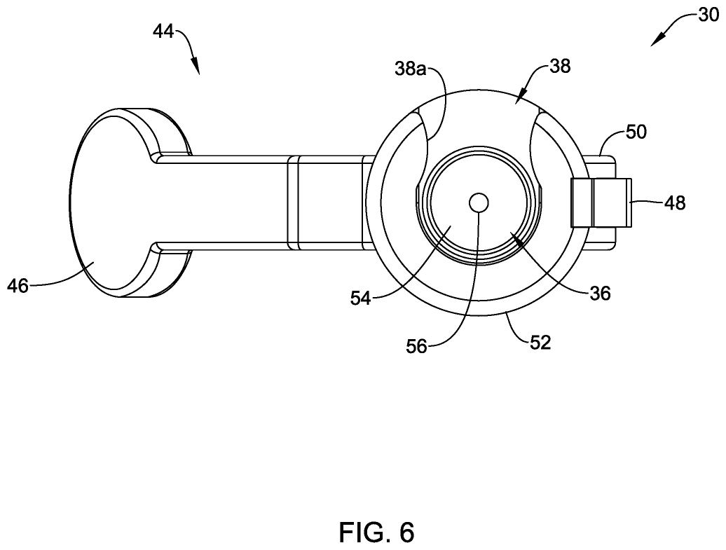

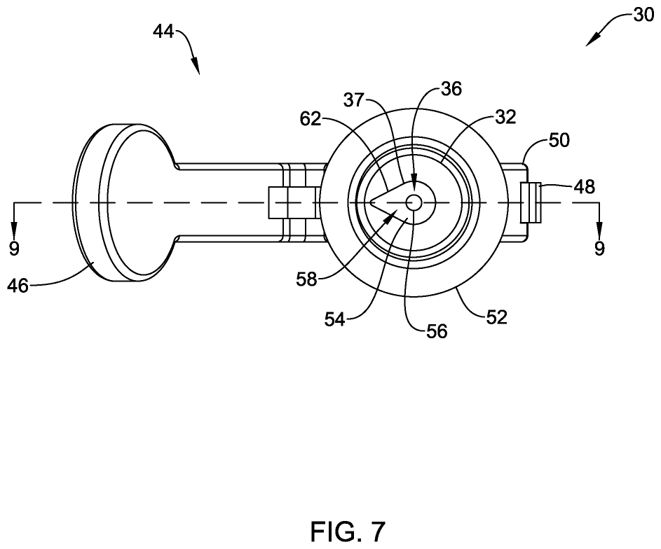

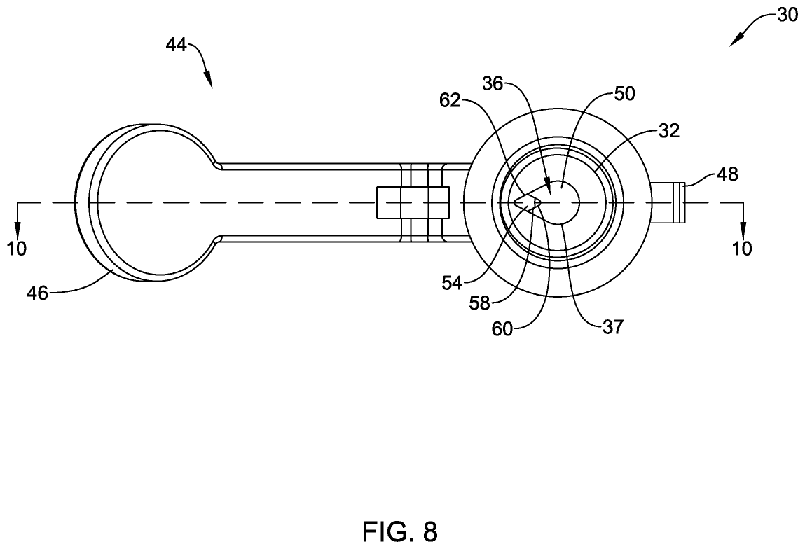

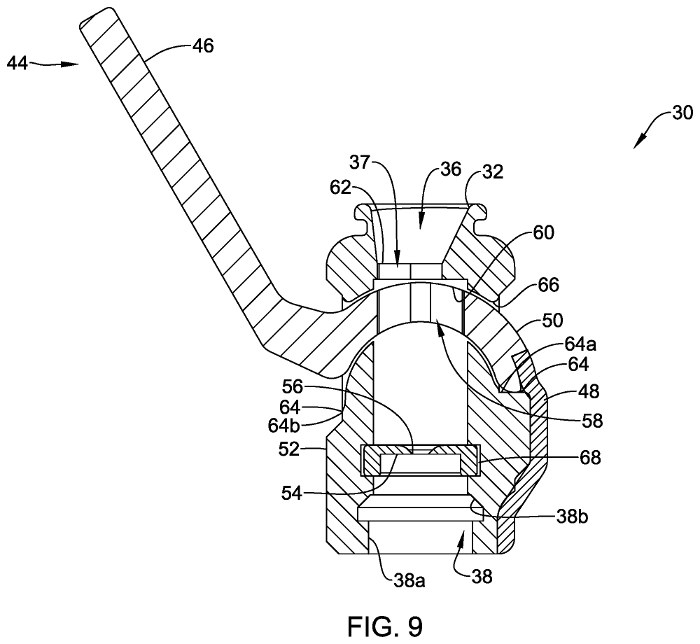

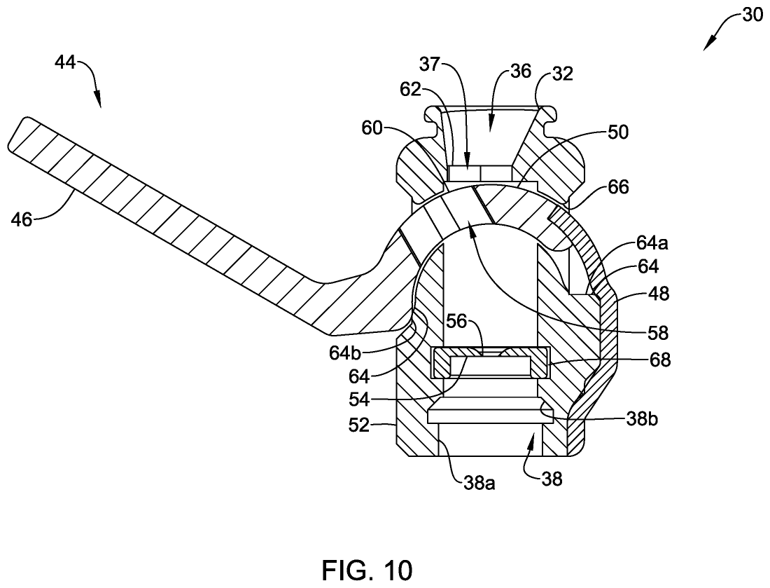

FIGS. 4-10 schematically depict views of an illustrative configuration of the hub 30. FIG. 4 schematically depicts a top perspective view of the hub 30. FIG. 5 schematically depicts a side view of the hub 30. FIG. 6 schematically depicts a bottom view of the hub 30. FIGS. 7 and 8 schematically depict top views of the hub 30 in the first configuration and the second configuration, respectively. FIGS. 9 and 10 schematically depict cross-section view of the hub 30 in the first configuration and the second configuration, taken along line 9-9 in FIG. 7 and line 10-10 in FIG. 8, respectively. Although the hub 30 may include additional and/or alternative components, the hub 30 may include the opening 32, the instrument channel 36, the connector 38, the actuator 44, the lever 46, the biasing member 48, a locking member 50, a housing 52, one or more seals or valves, and/or other suitable components.

The housing 52 may have any suitable configuration. In some examples, the housing 52 may be a body of the hub 30 and may define an outer or inner surface of one or more portions of the hub 30. In some examples and as depicted in FIGS. 4-10, the housing 52 may define at least part of an outer surface of the hub 30, an inner surface of the hub 30, the instrument channel 36, the opening 32, and the connector 38. In some examples, the inner surface of the housing 52 may entirely or at least partially define the connector 38, the opening 32, the instrument channel 36, and/or other suitable portions of the hub 30.

The lever 46 of the hub 30 may have any suitable configuration. The lever 46 may be configured to receive force from a user to adjust the locking member 50 to a closed configuration and/or an opened configuration. The locking member 50 may be a separate component form the actuator 44 and/or the locking member 50 or may be formed integrally with or otherwise be a portion of the actuator 44 and/or the locking member 50. Example suitable configurations of the lever 46 include, but are not limited to, an arm, a tab, a paddle, a button, an extension, and/or other suitable component in communication with the locking member 50. As depicted in FIGS. 4-10, the lever 46 may be an arm or extension of or integrally formed with the locking member 50. Other suitable configurations of the lever 46 are contemplated.

The locking member 50 of the hub 30 may have any suitable configuration designed to be in communication with the instrument channel 36 and configured to adjust between the first configuration and the second configuration. In some examples, the locking member 50 may be coupled with the lever 46 and/or the biasing member 48. Alternatively or additionally, the locking member 50 may be integrally formed with or part of the lever 46 and/or the biasing member 48. In some examples, a first end of the locking member 50 may be coupled with the biasing member 48 and a second end of the locking member 50 may be coupled with or may form the lever 46. As depicted in FIGS. 4-10, the locking member 50 may be integrally formed with the lever 46 and coupled with the biasing member 48.

The connector 38 of the hub 30 may have any suitable configuration configured to couple with the coupling portion 28 of the access port 20. In some examples and as depicted in FIG. 5, the connector 38 may include a first portion 38a configured to receive the neck 40 of the coupling portion 28 and a second portion 38b configured to receive the flange 42 of the coupling portion 28. In some examples, one or both of the first portion 38a and the second portion 38b of the connector 38 may have dimensions smaller than the neck 40 or the flange 42, respectively, such that the connector 38 creates a friction fit, clip connection, and/or snap connection with the coupling portion 28. Other suitable configurations of connector 38 configured to couple with the coupling portion 28 of the access port 20 using suitable types of connection or coupling techniques are contemplated.

The biasing member 48 may be any suitable type of biasing member. For example, the biasing member 48 may be one or more resilient materials, one or more stretchable materials, one or more springs, one or more elastic materials, and/or one or more other suitable components configured to bias the actuator 44 (e.g., the lever 46 and/or the locking member 50) to a configuration. In some examples and as depicted in FIGS. 4-10, the biasing member 48 may be an elongate component formed from elastic material that is configured to bias the locking member 50 to an opened configuration (e.g., a first configuration), but other suitable configurations of the biasing member 48 are contemplated for biasing the locking member 50 to the opened configuration. In other examples, the biasing member 48 may be configured to bias the locking member 50 a closed configuration.

FIG. 6 depicts a schematic bottom view of the hub 30 showing a seal 54 in the instrument channel 36. In some examples, the seal 54 may be positioned within the hub 30 so as to extend across the instrument channel 36 at one or more axial locations along the hub 30. In some examples, the seal 54 may be configured to open, grip, and/or seal around one or more medical instruments that are inserted in the instrument channel 36 and/or through the hub 30. In some examples, the seal 54 may include the seal opening 56 that may be configured to receive the one or more medical instruments inserted in the instrument channel 36. The seal 54 and/or the seal opening 56 in the seal 54 may be configured to prevent or mitigate an exchange of fluids across the seal 54 by “squeegeeing” liquids from an outer surface of instruments received in the seal opening 56 as the instruments are inserted or withdrawn from the hub 30 (e.g., across the seal 54). Although not depicted in FIG. 6, the seal opening 56 may be configured to substantially or completely close when unoccupied by an instrument to prevent or mitigate an exchange of fluids and/or insufflation loss.

The seal 54 may have any suitable configuration. Example suitable configurations of the seal 54 include, but are not limited to, the seal configurations described in or incorporated by reference in U.S. Pat. No. 12,070,187, filed on Nov. 1, 2019, titled DEVICES, SYSTEMS, AND METHODS FOR PROVIDING SEALABLE ACCESS TO A WORKING CHANNEL, which is hereby incorporated by reference in its entirety for any and all purposes, and U.S. Pat. No. 11,064,870, filed on Aug. 10, 2018, titled BIOPSY CAP FOR USE WITH ENDOSCOPE, which is hereby incorporated by reference in its entirety for any and all purposes. Other suitable configurations of the seal 54 are contemplated.

FIGS. 7 and 8 depict schematic top views of the hub 30 with the actuator 44, including the locking member 50 in a first, opened configuration and in a second, closed configuration, respectively. When the locking member 50 is in the first configuration, a lock opening 58 of the locking member 50 may be aligned with the channel opening 37 and/or the seal opening 56 to facilitate receiving and/or adjusting one or more instruments in and/or through the instrument channel 36, as depicted for example in FIG. 7. When the locking member 50 is in the second configuration, an edge 60 of the lock opening 58 of the locking member 50 may be at least partially offset from (e.g., extend across at least part of) the channel opening 37 and/or the seal opening 56 to facilitate engaging and/or securing one or more instruments received in the instrument channel 36, as depicted for example in FIG. 8.

The channel opening 37 may have any suitable configuration for receiving one or more instruments therethrough. In some examples, the channel opening 37 may be configured to facilitate receiving and/or adjusting one or more instruments when the actuator 44 and/or the locking member 50 are in an opened or first configuration, as depicted for example in FIG. 7, and locking or securing one or more of the received instruments when actuator 44 and/or the locking member 50 are in a closed or second configuration, as depicted for example in FIG. 8. Example suitable configurations of the channel opening 37 include, but are not limited to, circular configurations, slot configurations, slit configurations, a shape having a rounded or circular base that tapers as the channel opening 37 extends laterally from the base (e.g., a tear drop shape), a diamond shape, a star shape, and/or other suitable shapes. In one example, the channel opening 37 may have a central or axial base that is rounded or circular and that tapers to a point or rounded edge as the channel opening extends radially from the base (e.g. in a direction of the lever 46 or other suitable direction). Other suitable configurations of the channel opening 37 are contemplated.

The lock opening 58 in the locking member 50 may have any suitable configuration for receiving one or more instruments therethrough. In some examples, the lock opening 58 may be configured to facilitate receiving and/or adjusting one or more instruments when the actuator 44 and/or the locking member 50 are in an opened or first configuration, as depicted for example in FIG. 7, and locking or securing one or more of the received instruments when the actuator 44 and/or the locking member 50 are in a closed or second configuration, as depicted for example in FIG. 8. For example, lock opening 58 may be configured such that when the locking member 50 is in the opened or first configuration, the lock opening 58 may be aligned with the channel opening 37 to allow one or more instruments received therethrough to rotate and/or longitudinally adjust without or with limited interference from the locking member 50 and/or the housing 52 and when the locking member 50 is in the closed or second configuration, an edge of the lock opening 58 may engage one or more of the received instruments to lock or secure the engages instruments with an interference fit or other suitable engagement with an edge 62 of the channel opening 37 and prevent or mitigate unintended longitudinal or rotational movement of the engaged instrument(s). Example suitable configurations of the lock opening 58 include, but are not limited to, circular configurations, latch configurations, slot configurations, slit configurations, a shape having a rounded or circular base that tapers as the channel opening 37 extends laterally from the base (e.g., a tear drop shape), a diamond shape, and/or other suitable shapes. In one example, the lock opening 58 may have a diamond shape with a central portion of the diamond shape may be configured to be aligned with (e.g., centrally aligned with) the base or widest portion of the channel opening 37 and the seal opening 56 when the locking member 50 is in the opened or first configuration and a corner portion of the diamond shape may extend across a widest portion (e.g., the base portion) of the channel opening 37 and/or the seal opening 56 when the locking member 50 is in the closed or second configuration. In some examples, the structure of the locking member 50 surrounding the lock opening 58 may block a portion of the channel opening 37 aligned with the seal opening 56 when the locking member is in the closed or second configuration. Other suitable configurations of the channel opening 37 are contemplated.

FIGS. 9 and 10 depict schematic cross-section views, taken along line 9-9 in FIG. 7 and line 10-10 in FIG. 8, respectively, of the hub 30 with the actuator 44, including the locking member 50 in a first, opened configuration and in a second, closed configuration, respectively. When the locking member 50 is in the first configuration, a lock opening 58 of the locking member 50 may be aligned with the channel opening 37 and/or the seal opening 56 to facilitate receiving and/or adjusting one or more instruments in and/or through the instrument channel 36, as depicted for example in FIG. 9. When the locking member 50 is in the second configuration, the edge 60 of the lock opening 58 of the locking member 50 may be at least partially offset from (e.g., may extend across at least part of) the channel opening 37 and/or the seal opening 56 to facilitate engaging and/or securing one or more instruments received in the instrument channel 36, as depicted for example in FIG. 10.

The housing 52 may define, or the hub 30 may otherwise include, one or more hard stops 64. In some examples, the locking member 50 may be configured to engage the hard stop 64 when the actuator 44 and/or the locking member 50 are in the first configuration and/or when the actuator 44 and/or the locking member 50 are in the second or closed position. In one example, the biasing member 48 may be coupled with the locking member 50 such that the locking member 50 is biased against the hard stop 64. In one example and as depicted in the configuration of the hub 30 in FIGS. 9 and 10, a first hard stop 64a may be positioned at a location of the housing 52 such that the biasing member 48 biases an end of the locking member 50 to engage the hard stop 64 when the locking member 50 is in the first, opened configuration and a second hard stop 64b may be positioned to stop movement of the locking member 50 when the locking member 50 reaches the second, closed configuration. Other suitable configurations of the hard stop relative to the configurations of the locking member 50 and the biasing member 48 are contemplated.

The locking member 50 may have any suitable configuration as part of or extending from the actuator 44 or lever 46 and configured to adjust between the first configuration and the second configuration when a user applies force to the actuator 44 and/or the lever 46. Example, suitable configurations of the locking member 50 include, but are not limited to, arc-shapes, rounded shapes, elongate shapes, planar shapes, valves, slides, and/or other suitable configurations. In one example and as depicted in FIGS. 9 and 10, the locking member 50 may include the lock opening 58 and may have an arced or arched cross-section.

The housing 52 of the hub 30 may include a lock adjustment opening 66 configured to receive the locking member 50 as the locking member 50 adjusts between the first configuration and the second configuration. In some examples, the lock adjustment opening 66 may be configured to receive the locking member 50 and allow for adjustment of the locking member 50 between the first configuration and the second configuration. In one example, when the locking member 50 is arced or arched, the lock adjustment opening 66 may be arced or arched, as depicted for example in FIGS. 9 and 10.

The housing 52 may include one or more seal locators 68. In some examples, the one or more seal locators 68 may include or be an indentation or protrusion in an inner surface of the housing 52 and configured to receive or otherwise engage the seal 54 at a desired axial location along the instrument channel 36. In one example and as depicted in FIGS. 9 and 10, the seal locator 68 may be an indentation or recess (e.g., an annular indentation or recess, etc.) in the housing 52 configured to receive and maintain the seal 54 at a desired axial location along the instrument channel 36. Other suitable configurations of the seal locator(s) 68 are contemplated.

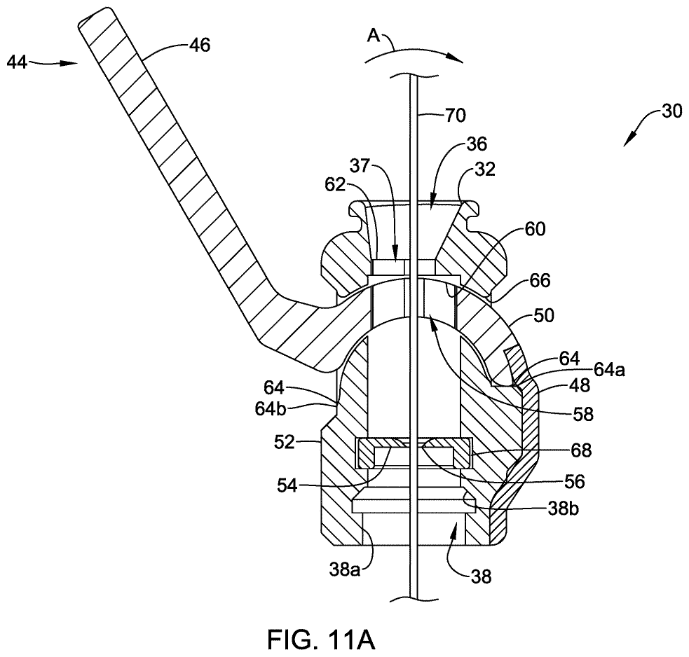

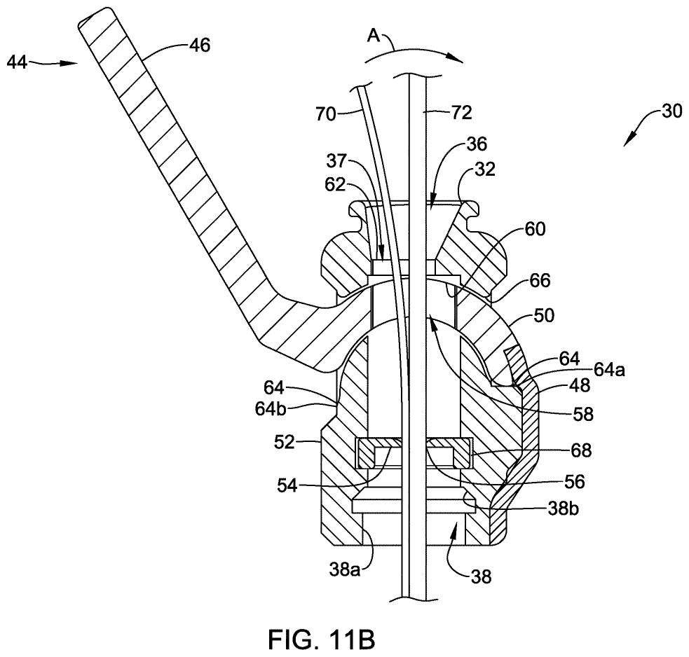

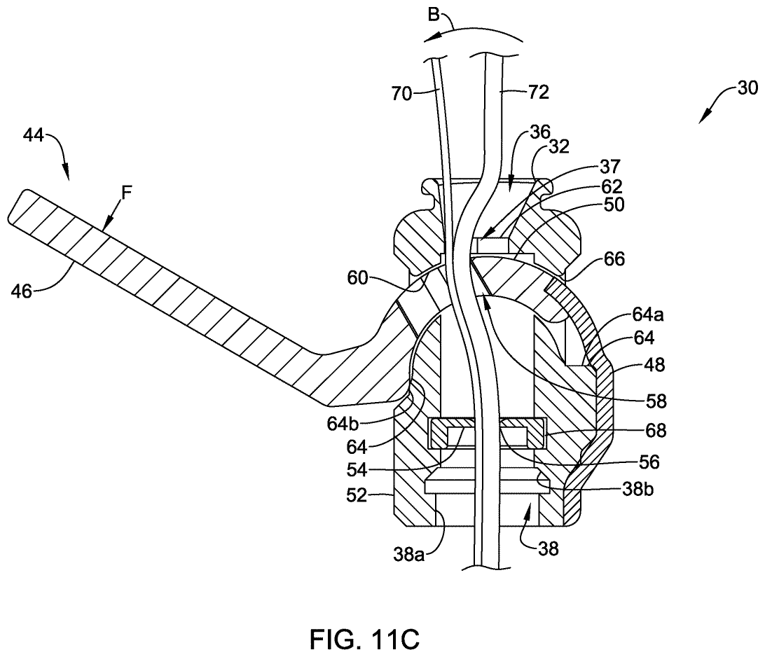

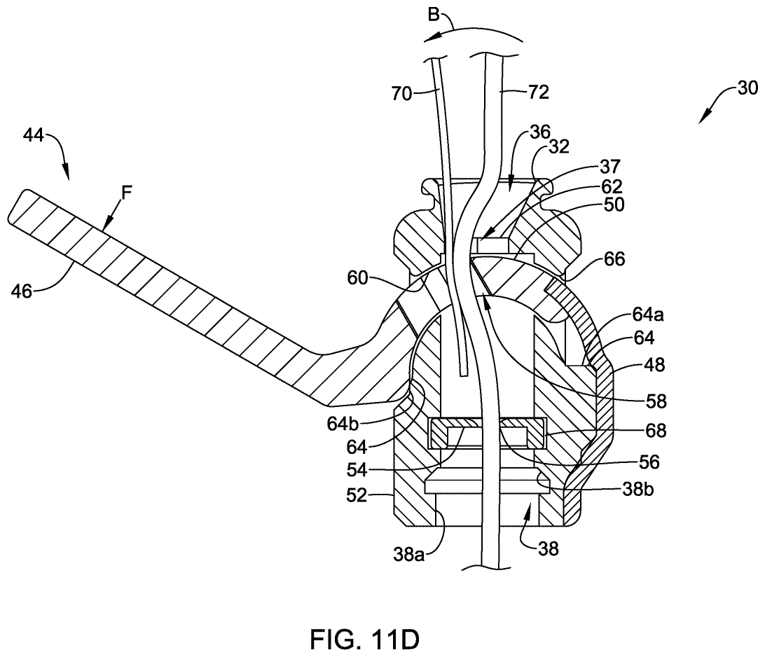

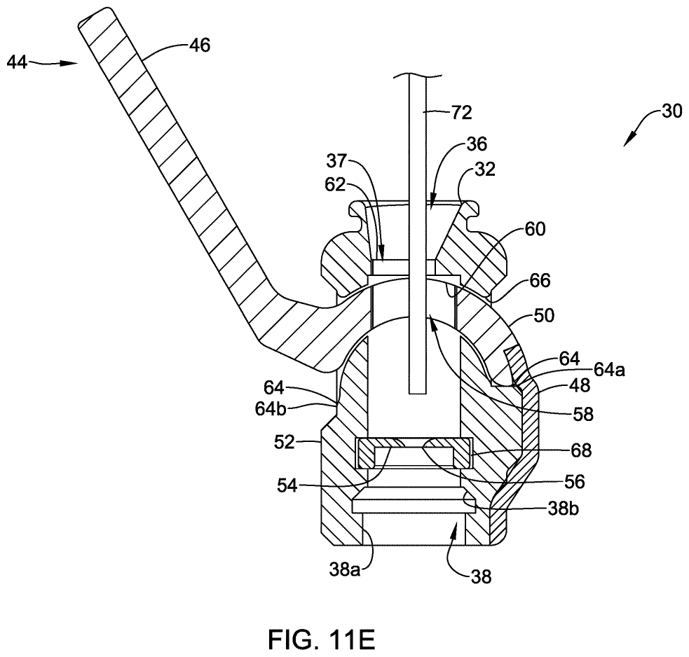

FIGS. 11A-11E schematically depict cross-section views of the hub 30 used in a method of receiving and engaging one or more instruments. Although the method is depicted and described with respect to the configurations of the hub 30 of FIGS. 4-10, other suitable configurations of the hub 30 may be used with the method. Although the hub 30 is not depicted in FIGS. 11A-11E as being part of or coupled with an endoscope, the hub 30 may be part of or coupled with an endoscope such that the instrument channel 36 is in communication with a working channel or other suitable channel of the endoscope.

The hub 30 may be configured to receive and facilitate delivery and/or exchange of a variety of medical instruments through the access port 20 and into the working channel 16 of an endoscope, laparoscope, or other visualization systems such as the Spy Glass™ Direct Visualization System (Boston Scientific Corp., Marlborough, MA). The hub 30 may be adapted to fit and/or be used with a variety of medical instruments and medical applications. Example medical instruments include, but are not limited to, sutures, cannulas, needles, catheters, aspiration devices, scopes and/or other suitable medical instruments.

FIG. 11A schematically depicts a cross-section view of the hub 30 with the locking member 50 in a first, opened configuration. In some examples, the biasing member 48 may bias the locking member 50 in a first direction A to the first, opened configuration, but other suitable configurations are contemplated. In the opened configuration, the base portion of the channel opening 37, a widest potion of the lock opening 58, and the seal opening 56 may be axially aligned and may at least partially define the instrument channel 36. As depicted in FIG. 11A, a first instrument 70 may be inserted through the opening 32 of the hub 30 and through the instrument channel 36 and into a working channel of an endoscope when the hub 30 is part of or coupled with the endoscope. Inserting the second instrument 72 through the instrument channel 36 may include inserting the second instrument 72 through one or more of (e.g., each of) the channel opening 37, the lock opening 58, and the seal opening 56. In some examples, the first instrument 70 may be a suture, but the first instrument 70 may be one or more other suitable instruments. When the first instrument 70 is extending through the instrument channel 36, the first instrument may be axially and/or rotationally adjusted, as desired.

FIG. 11B schematically depicts a cross-section view of the hub 30 with the locking member 50 in the first, opened configuration, as depicted in FIG. 11A. With the first instrument 70 inserted through the instrument channel 36, a second instrument 72 may be inserted through the opening 32 of the hub 30 and through the instrument channel 36. Inserting the second instrument 72 through the instrument channel 36 may include inserting the second instrument 72 through one or more of (e.g., all of) the channel opening 37, the lock opening 58, and the seal opening 56. In some examples, the second instrument 72 may be a cannula or catheter or other suitable instrument having a diameter greater than the first instrument 70. In some examples, the second instrument 72 may be an instrument having a diameter that is larger than the diameter of the first instrument 72 such that the hub may lock or engage the second instrument 72 in a desired position while allowing the first instrument to be adjustable.

Although FIGS. 11A and 11B depict the first instrument 70 inserted before the second instrument 72, the second instrument 72 may be inserted before the first instrument 70 or the first instrument 70 and the second instrument 72 may be inserted into the hub 30 and/or through the instrument channel 36 at the same time (e.g., simultaneously). Additionally or alternatively to using a single first instrument 70 and a single a second instrument 72, as depicted in FIG. 11B, additional or other instruments may be inserted into the hub 30 and/or through the instrument channel 36.

FIG. 11C schematically depicts a cross-section view of the hub 30 with the locking member 50 in a second, closed configuration. In some examples, with the first instrument 70 and the second instrument 72 extending through the channel opening 37 and the lock opening 58, a user may apply a force F to the lever 46 with a finger of a hand holding the endoscope including or coupled with the hub 30 or with one or more other fingers, which in-turn applies a force to the locking member 50, to adjust the locking member 50 in a second direction B from the first, opened configuration to the second, closed configuration, but other suitable configurations are contemplated. Such locking of the second instrument 72 in-place with a hand holding the endoscope may allow the medical practitioner to control the endoscope and/or adjust the first instrument 70 with the other hand of the medical practitioner. As such, a single medical practitioner may be able to maintain a position of an endoscope while also performing a procedure using the endoscope.

In the closed configuration, the edge 60 of the lock opening 58 may engage the second instrument 72 and push the second instrument 72 against a portion of the edge 62 of the channel opening 37 along or defining the tapering portion of the channel opening 37. The engagement of the second instrument 72 between the edge 60 of the lock opening 58 and the edge 62 of the channel opening 37 may create an interference fit and longitudinally and/or rotationally lock the second instrument 72 in a desired position relative to the working channel of the endoscope (e.g., to prevent or mitigate longitudinal and/or rotational movement of the second instrument 72). Due to the shape or configuration of the channel opening 37 and the lock opening 58 and the first instrument 70 having a diameter smaller than the diameter of the second instrument 72, the first instrument 70 may be rotationally and/or longitudinally adjusted relative to the working channel of the endoscope while the second instrument 72 is secured in the desired position. FIG. 11D schematically depicts an example of the first instrument 70 being removed from the hub 30 or adjusted longitudinally in a proximal direction relative to the hub 30, while maintaining a position of the second instrument 72, with the locking member 50 in the second configuration. In some examples, with the locking member 50 in the second configuration, one or more first instruments 70 may be inserted into and/or through the instrument channel 36. In some examples, the first instrument 70 may be a suture and the second instrument 72 may be a cannula that needs to remain in a desired position as the suture is adjusted, but other suitable configurations are contemplated.

When the second instrument 72 is to be removed from the hub 30 and/or adjusted relative to the working channel of the endoscope and/or in other instances, the locking member 50 may be adjusted to the first, opened configuration, as depicted for example in FIG. 11E. For example, a user may release or otherwise remove the force F from the lever 46, and thus from the locking member 50, and the biasing member 48 may automatically cause the locking member 50 to return the first, opened configuration. With the locking member 50 in the first, opened configuration, the second instrument 72 may be adjusted relative to the hub 30 and/or removed from the hub 30.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A hub configured for use with an endoscope, the hub comprising:
a housing defining a channel configured to be in communication with a working channel of the endoscope and receive one or more instruments; and
a locking member in communication with the channel and configured to adjust between a first configuration and a second configuration,
wherein the locking member is configured to engage an instrument of the one or more instruments and prevent longitudinal movement of the instrument when the locking member is in the second configuration and the locking member is configured to allow the instrument to adjust longitudinally when the locking member is in the first configuration.

2. The hub of claim 1, further comprising:
a biasing member configured to bias the locking member in the first configuration.

3. The hub of claim 1, further comprising:
a biasing member configured to bias the locking member in the second configuration.

4. The hub of claim 1, wherein the locking member comprises an opening configured to receive the instrument when the locking member is in the first configuration.

5. The hub of claim 4, wherein the locking member is configured to receive a second instrument of the one or more instruments in the opening when the locking member is in the first configuration and allow the second instrument to longitudinally adjust when the locking member is in the second configuration.

6. The hub of claim 1, wherein the housing is configured to releasably couple with a port of the endoscope.

7. The hub of claim 1, wherein the housing comprises an opening configured to receive a port of the endoscope and axially align the channel with an opening of the port.

8. The hub of claim 1, further comprising:
a biasing member configured to bias the locking member in one of the first configuration and the second configuration,
wherein the locking member comprises a lever and is coupled with the biasing member.

9. The hub of claim 8, wherein a first end portion of the locking member is coupled with the biasing member and a second end portion of the locking member forms the lever configured to receive a force from a user to adjust the locking member from the first configuration to the second configuration.

10. The hub of claim 1, wherein the locking member has an arc portion extending through the channel.

11. The hub of claim 1, wherein the locking member extends through the channel and includes an opening in communication with the channel.

12. The hub of claim 11, wherein the opening has a first central axis aligned with a second central axis of the channel when the locking member is in the first configuration and the first central axis is offset from the second central axis when the locking member is in the second configuration.

13. The hub of claim 1, further comprising:
a seal located in the channel,
wherein the seal is configured to receive the one or more instruments.

14. An endoscope comprising:
a handle comprising an instrument port defining an instrument channel configured to receive one or more instruments therein;
an elongate shaft extending in a distal direction from the handle and defining a lumen in fluid communication with the instrument channel; and
a locking member in communication with the instrument channel and configured to adjust between a first configuration and a second configuration,
wherein the locking member is configured to engage an instrument of the one or more instruments and prevent longitudinal movement of the instrument when the locking member is in the second configuration and the locking member is configured to allow the instrument to adjust longitudinally when the locking member is in the first configuration.

15. The endoscope of claim 14, further comprising:
a biasing member,
wherein the locking member comprises a lever coupled with the biasing member and configured to engage the instrument of the one or more instruments when the locking member is in the second configuration.

16. The endoscope of claim 14, wherein the instrument port includes a hub housing configured to releasably couple with a handle housing coupled with the elongate shaft.

17. The endoscope of claim 16, wherein the hub housing is configured to clip onto the handle housing.

18. A method, the method comprising:
inserting a first instrument through an opening in a hub of an endoscope when a locking member of the hub is in a first configuration;
inserting a second instrument through the opening in the hub; and
adjusting the locking member to a second configuration to engage the first instrument with the locking member and prevent longitudinal movement of the first instrument while the second instrument is longitudinally adjustable.

19. The method of claim 18, further comprising:
inserting the first instrument and the second instrument through a lock opening in the locking member,
wherein the first instrument is engaged by an edge of the opening in the hub and an edge of the lock opening when the locking member is in the second configuration.

20. The method of claim 18, wherein adjusting the locking member to the second configuration comprises applying a force on the locking member and the method further comprises:
releasing the locking member to cause the locking member to automatically return to the first configuration.
